(19) United States
(12) Patent Application Publication
(10) Pub. No.: US 2026/0090979 A1
(43) Pub. Date: Apr. 2, 2026

(54) COSMETIC USES OF A HYDROLYSATE OF AN EXTRACTION COPRODUCT OF THE MICROALGAE PHAEODACTYLUM TRICORNUTUM

(71) Applicant: MICROPHYT, Baillargues (FR)

(72) Inventors: Cyrielle HOUDIN, Montpellier (FR); Rémi PRADELLES, Montpellier (FR); Clémence THEBAUD, Castelnau Le Lez (FR)

(73) Assignee: MICROPHYT, Baillargues (FR)

(21) Appl. No.: 19/111,182

(22) PCT Filed: Sep. 20, 2023
(86) PCT No.: PCT/FR2023/051436

§ 371 Date: Mar. 12, 2025

(30) Foreign Application Priority Data
Sep. 21, 2022 (FR) FR2209544

(51) Int. Cl. A61K 8/9706; A61Q 19/00

(52) U.S. Cl. CPC A61K 8/9706; A61Q 19/00; A61K 2800/92

(57) ABSTRACT

A cosmetic or nutraceutical use of a hydrolysate of an extraction coproduct of the microalgae Phaeodactylum tricornutum to exfoliate skin and/or to reduce skin microrelief. A method of cosmetic care includes the topical application of a hydrolysate of an ethanolic extraction coproduct of the microalgae P. tricornutum or of a composition including it to exfoliate skin and/or to reduce skin microrelief.

The invention is related to the use of a hydrolysate of an extraction coproduct of the microalgae Phaeodactylum tricornutum to exfoliate skin and/or to reduce skin microrelief.

The skin is an organ made of several layers: epidermis, dermis and hypodermis. The epidermis represents the superficial layer and is also itself made of several layers, the horny layer (stratum corneum), the clear layer (stratum lucidum), the granular layer (stratum granulosum), the Malpighi layer and the basal lamina (stratum basale) corresponding the deepest layer, biosynthesis site of keratinocytes. The horny layer represents the most superficial layer. It contains notably the sweat glands and skin appendages (hair, hairs) but it is also the layer which accumulates dead cells, the desquamation site as such. Desquamation is a natural mechanism generating squama, corneocyte clusters. It is also qualified of exfoliation. This mechanism is entirely part of cell renewal.

Mechanical solutions (peeling, exfoliating, circular massages thanks to formulations containing synthetic or natural abrasive grains such as grounded fruit kernels or seeds, sugar, seeds) in order to exfoliate skin are widely used in the cosmetic domain but these solutions are aggressive for skin, they are abrasive, and can generate redness, irritation, tingling or warmth. They are not well suited in particular for sensitive skin, neither to acneic skin or skin prone to acne.

Few natural exfoliating ingredients are available onto the cosmetic market. Natural fruit extracts such as extracts from kiwi, lemon, honey or also glycolic acid that can be found notably in sugar cane, sugar beet or grape can be cited. But the fruit acids which are found in these natural extracts (alphahydroxy acids and betahydroxy ie. AHA, BHA) are aggressive for the skin. Retinoids are also known as exfoliating agents but they can also be aggressive for the skin.

Therefore, there is a need in the cosmetic domain of alternative active ingredients capable of exfoliating the skin in a natural way, without mechanical intervention, allowing to eliminate cell debris, or squama, to promote cell renewal without stripping the superficial layers of the epidermis and to promote a gentle and continuous exfoliating, what fruit acids do not allow.

Surprisingly, the Applicant discovered that a hydrolysate of an extraction coproduct of Phaeodactylum tricornutum showed efficacy onto skin exfoliation as well as an action of reducing skin microrelief.

One of the advantages of the hydrolysate according to the invention is that it is obtained from a natural coproduct of extraction of a microalga, non-genetically modified, without hormone supplementation or chemical compounds. It is easily obtained industrially, in a sustainable way, notably in photobioreactors by autotrophic culture without impact onto arable lands. The hydrolysate is not irritative for the skin, not sensitizing.

The hydrolysate according to the invention comes from a culture of the microalga P. tricornutum. Phaeodactylum tricornutum is a diatom part of the Phaeodactylum genus and the Phaeodactylaceae family, found under 3 distinct morphotypes (spindle-shaped, oval, triradiate) found in many oceanic regions of the world, including northern Europe, Oceania and the Atlantic.

Extracts of P. tricornutum are already known for their use in the nutraceutical domain. An extract of P. tricornutum in a composition as a food supplement in order to improve cognitive capacities of aged individuals or presenting cognitive decline is so known from the Applicant. Extracts of P. tricornutum for their cosmetic uses are also described. In this respect, patent application US2004136945 describes a cosmetic care process comprising the topical application of an effective amount of a P. tricornutum extract obtained by extraction with a polar solvent to improve skin firmness and elasticity and reduce wrinkles.

Patent application US2010092506 describes the use in a cosmetic composition of an extract of P. tricornutum as a depigmenting active agent, intended to attenuate or eliminate pigmentation spots on the skin or lighten hair or hairs complexion.

Patent application WO2020178213 relates to a non-therapeutic care method comprising the application of a cosmetic composition comprising a hydrophilic extract of P. tricornutum to reduce redness in sensitive skin, to soothe and to reduce inflammation in sensitive skin. Said composition is also effective in improving cutaneous microbiota. However, this application does not disclose the use of a hydrolysate of a P. tricornutum extraction coproduct for exfoliating skin and/or reducing skin microrelief.

Thus, to the best of the Applicant's knowledge, no prior art describes or suggests the use of a hydrolysate of P. tricornutum extraction coproduct to exfoliate the skin or to reduce skin microrelief.

A first object of the present invention relates to the cosmetic use of a hydrolysate of a coproduct from an extraction of P. tricornutum (ie. hydrolysate of an extraction coproduct of P. tricornutum) to exfoliate skin and/or reduce skin microrelief. Another object relates to the cosmetic use of the hydrolysate in a composition. Yet another object relates to a cosmetic care process comprising the topical application of a hydrolysate of an extraction coproduct of P. tricornutum or a cosmetic composition comprising it, to exfoliate skin and/or to reduce skin microrelief.

The term «cosmetic use» means a non-therapeutic use, ie. non-pharmaceutical and non-dermatological use.

The term “healthy skin” means any area of skin qualified as non-pathological by a dermatologist, i.e. an area of skin free from injury, redness, infection, scarring, allergy, wound, disease, eczema, inflammation, acne and/or dermatitis.

The term “skin” means all or part of the face and/or body selected from any area of the arms, including forearms, legs, thighs, back, torso, neck, neckline, feet and/or hands. For the purpose of the invention, skin includes the scalp.

The hydrolysate according to the invention is administered orally or applied topically, advantageously applied topically.

The term “topical route” means the vaporization of the hydrolysate onto the surface of the skin, or the direct local application onto the skin. The hydrolysate or the composition comprising it is topically acceptable. By “topically acceptable” it is meant a hydrolysate or composition which does not induce an allergic reaction on the skin, and which is not irritating.

The term “coproduct” means the residue of any extraction of the biomass of the photosynthesizing microalga P. tricornutum, obtained by a process enabling the hydrolysate according to the invention to be obtained indirectly, as described further in this application. The term “coproduct” will be used preferentially in the description. By “hydrolysate” it is meant any product resulting from a hydrolysis, as described further in this application.

In the present application, the term “expression” means gene expression (expression of complementary cDNAs) and/or protein expression, it being understood that protein expression includes the expression of messenger RNAs (mRNAs) within the meaning of the invention.

By “exfoliating the skin”, it is meant naturally increasing the amount of squama released from the surface of the upper layer of the epidermis. The term “naturally” also refers to a biological action, as opposed to a mechanical action, particularly of the abrasive type, such as that exerted by sea salt or silica grains.

In one embodiment of the invention, “exfoliating the skin” means increasing by at least 5%, advantageously by at least 6%, very advantageously by at least 6.5% the surface area of the skin, preferably healthy skin, occupied by squama in the upper layers of the epidermis in the presence of the hydrolysate according to the invention, compared with the surface area occupied by squama without the hydrolysate according to the invention (Placebo).

Advantageously, it is an increase in the surface area of the upper layer of the epidermis occupied by squama after application to the forearm of a population of 35 healthy individuals, twice a day for 28 days, of a cream comprising 1.5% by weight, relative to the total weight of the cream, of the hydrolysate according to the invention in comparison with a placebo cream, under the conditions described in Example 2. Even more advantageously, said surface area is measured in mm². Very advantageously, the hydrolysate according to the invention included in the cream is a hydrolysate of the coproduct of the ethanolic extraction of P. tricornutum as prepared under the conditions of Example 1a). Results are presented in Table 1.

In another embodiment of the invention, “exfoliating the skin” means increasing by at least 10%, advantageously by at least 20% in the presence of the hydrolysate according to the invention compared with placebo, the desquamation index (ID) of samples of the upper layers of the epidermis, said index being defined as the sum of twice the total surface area occupied by squama (mm²) (A below) and the sum of the percentage of layers in relation to the thickness of each layer, the number of layers being equal to 5, according to the following formula as described by Schatz et al. (1993) [Schatz H., Kligman A M., Manning S., Stoudemayer T. (1993) Quantification of dry (xerotic) skin by image analysis of scales removed by adhesive discs (D-Squames). J. Soc. Cosmet. Chem. 44, 53-63], under the conditions described in Example 2:

$$ID = [ 2 \times A + \text{?} n \times ( n - 1 ) ] / 6 \qquad [ Math 1 ]$$

$$\text{?} \text{indicates text missing or illegible when filed}$$

where A represents the total surface area occupied by scales (mm²), Tn represents the percentage of layers in relation to the thickness of each layer, and n represents the thickness from 1 to 5.

Advantageously, the increase in the desquamation index is measured within the framework of the clinical study described above and which protocol is detailed in Example 2, i.e. within a population of 35 individuals, advantageously healthy i.e. not suffering from any skin pathology and presenting healthy skin as defined in the present invention, after application twice a day for a period of 28 days of the cream comprising 1.5% by weight, relative to the total weight of the cream, of the hydrolysate according to the invention in comparison with the placebo cream.

Even more advantageously, the hydrolysate according to the invention included in the cream is the hydrolysate of the coproduct from the ethanolic extraction of P. tricornutum as prepared under the conditions of Example 1a). The results are shown in Table 2 of Example 2. Very advantageously, the desquamation index is measured after sampling the epidermal surface layers of the population concerned, at times D14 and D28 (14 days and 28 days), after application for a period of 20 seconds onto the forearms of the population of a patch consisting of a transparent acetate disk and an adhesive film (D'Squame®). The samples are then analyzed using QuantiSquam® image acquisition software, under the conditions described in Example 2, with the amount of desquamated epidermal material increasing as the light is whiter (Schatz et al. (1993), cited above). The results are shown in Table 2 of Example 2 and in [FIG. 1].

The hydrolysate according to the invention is effective for exfoliating the skin, and is therefore useful for increasing skin cell renewal, preferably in healthy skin.

Also for the purpose of the invention, “reducing skin microrelief” means smoothing the skin in the presence of the hydrolysate according to the invention. In one embodiment of the invention, this means a reduction in the surface heterogeneity of the skin, preferably healthy skin, in the presence of the hydrolysate according to the invention. Advantageously, skin heterogeneity is measured as part of the clinical study described above in a population of 35 individuals after twice-daily application, for a period of 28 days, of a cream comprising 1.5% (w/w) of the hydrolysate according to the invention, or of a placebo cream (without hydrolysate).

Thus, in this embodiment, the hydrolysate according to the invention is considered to be in an effective amount for reducing skin microrelief when the reduction in skin surface heterogeneity measured after 28 days of application of the cream comprising the hydrolysate is at least 1%, preferably at least 2%, still more preferably at least 2.5%, compared with the skin surface heterogeneity measured at time TO of the start of the clinical study conducted. Preferably, the hydrolysate according to the invention included in the cream is the hydrolysate of the ethanolic extraction coproduct of P. tricornutum as prepared under the conditions of Example 1a). Even more advantageously, the surface heterogeneity is measured via topical application of a silicone resin (Silflo®) onto the forearms of the population concerned at time D28 (28 days of application of the cream of interest), which is applied for a period of 8 minutes in order to harden. The cured resin is then removed from the skin to form a faithful negative replica of the skin area concerned. The replica is analyzed by image analysis using Quantilines® (Monaderm) software (Parameter Rt), under the conditions described in the protocol of Example 3.

In an alternative embodiment of the invention, “reducing skin microrelief” means reducing the roughness parameter Rz of the preferably healthy skin of the forearms of the population described in the above clinical study, in the presence of the hydrolysate according to the invention, advantageously the hydrolysate of the ethanolic extraction co-product of P. tricornutum as prepared under the conditions of Example 1a), by at least 1.5%, advantageously by at least 3% at the end of 28 days (D8) compared with the parameter Rz measured onto the forearms to which the cream comprising the hydrolysate was applied at time 0 (DO). Parameter Rz enables skin roughness to be measured using the same Quantilines® software (Monaderm), after application at time D28 of the silicone resin (Silflo®) as described above, under the conditions described in Example 3.

In another alternative embodiment of the invention, “reducing skin microrelief” means reducing the parameter Ra related to skin relief, preferably healthy skin, of the forearms of the population described in the above clinical study, in the presence of the hydrolysate according to the invention, advantageously the hydrolysate of the ethanolic extraction co-product of P. tricornutum as prepared under the conditions of Example 1a), by at least 1.5%, advantageously by at least 2.5% at the end of 28 days (D8) compared with the parameter Ra measured onto the forearms onto which the cream comprising the hydrolysate was applied at time 0 (D0). The software Quantilines® (Monaderm), following application at D28 of the silicone resin (Silflo®) as described above, enables the measurement of the parameter Ra, under the conditions described in Example 3. Results of the reduction of parameters Rt, Rz and Ra in the presence of the hydrolysate according to the invention are presented in Example 3 and [FIG. 2].

The microalgae P. tricornutum is cultivated under controlled conditions in suitable systems such as race-ways, open ponds or closed systems such as photobioreactors. Photobioreactors used can be of any existing type such as horizontal tubular photobioreactors, vertical such as systems qualified as “green wall panel”, flat photobioreactors or in column. Preferentially, biomass production takes place in a closed culture photobioreactor.

The microalgae culture can be operated under batch culture mode, fed-batch, continuous, semi-continuous, turbidostat or chemostat.

The biomass obtained following culture can be concentrated by discarding all or part of the water using chemical or physical processes such as centrifugation, filtration, flocculation, sedimentation, associated or not to steps of drying by lyophilization, vacuum drying, drum drying, atomization or any other process that reduces the water content of the biomass. Cell lysis processes can then be used, such as the application of pressure, electrical flux or shear forces, the use of enzymes, or any other process enabling tissue destructuration, organs, cells or organelles.

The hydrolysate according to the invention is a hydrolysate of the residue, also named “coproduct”, said coproduct corresponding to the delipidated fraction obtained from any solid-liquid extraction process, which can be by supercritical or hypercritical or subcritical fluids, involving or not parallel or sequential microwave, ultrasound, pressure, or enzymatic cotreatments.

The biomass obtained following culture can be extracted fresh or can be dried before extraction. It is meant by “dried biomass” a microalgal biomass dehydrated comprising less than 15%, advantageously less than 10%, still advantageously less than 5% of water. The biomass can be centrifuged and then filtered to discard water before extraction. Advantageously, the biomass extraction is a solid-liquid extraction using a solvent or a mixture of solvent chosen from water, acetone, hexane, ethyl acetate, methyl tetrahydrofuran, 2-methyloxolan, heptane, an alcohol chosen from ethanol, methanol, isopropanol, a natural or branched oil, a glycol, a polyol, a mixture water/alcohol or a mixture water/glycol in the range 99/1 to 1/99 (w/v).

The biomass solid-liquid extraction is executed from a biomass quantity from 0.1 to 20% in weight, advantageously from 1% to 20%, more advantageously from κ% to 15%, and still more advantageously of 10% in weight compared to the total volume of the biomass and the extraction solvent.

The extraction can be executed under a temperature from 4° C. to 300° C., included room temperature, meaning of a temperature of 20° C.

In one embodiment of the invention, the biomass extraction is made in water under subcritical conditions, under a temperature from 100° C. to 300° C., advantageously from 120° C. to 250° C., more advantageously of 120° C. The extraction can be made under a fixed temperature or under increasing subsequent temperatures. In an advantageous embodiment of the invention, the extraction is made under a temperature of 120° C. In an alternative embodiment, it will be conducted under an increasing gradient of 3 temperatures comprised between 100° C. and 200° C., such as 120° C., 140° C. and then 160° C., or 110° C., 130° C. and then 150° C., or still 120° C., 145° C. and then 170° C.

It is meant by extraction under “subcritical conditions” an extraction with water, under conditions of temperatures above 100° C. and a pressure below 221 bar, the water being under liquid state at room temperature but having a viscosity and a surface tension below those of water at room temperature, therefore increasing its dielectric value. Therefore, the extraction pressure is comprised between 150 bar and 250 bar, preferentially between 200 and 221 bars, advantageously inside an extraction pressurized autoclave.

In another embodiment of the invention, the biomass extraction is conducted by supercritical carbon dioxide extraction. In such a case, said extraction can be conducted together with a co-solvent chosen from ethanol, acetone, methanol or any of their mixture. Alternatively, the carbon dioxide supercritical extraction can be conducted without co-solvent and followed by a further extraction with a polar solvent chosen from ethanol, acetone, methanol, hexane, heptane, advantageously ethanol.

Advantageously, the carbon dioxide supercritical extraction is conducted together with ethanol (10%) as co-solvent. The conditions of the carbon dioxide supercritical extraction are such that the pressure measured in bar is above 74 bar, advantageously above 100 bar, still advantageously above 150 bar. The temperature is above 35° C., advantageously above 45° C., still advantageously above 50° C. The delipidated fraction such obtained corresponds to the extraction coproduct described above.

It is further meant by “hydrolysate” any product obtained from the hydrolysis of the delipidated fraction. Advantageously, the hydrolysate is obtained by hot hydrolysis. It is meant here by “hot hydrolysis” a hydrolysis at a temperature of at least 35° C., advantageously of at least 40° C., still advantageously of at least 55° C. and more advantageously of 60° C.

The hydrolysis can be conducted under basic or acid conditions. It is conducted advantageously under basic conditions. It is meant here by “basic conditions” a hydrolysis driven at a pH comprised between 7.0 and 11.5, advantageously between 10 and 11, more advantageously a pH of 10.0. Advantageously, the hydrolysis is conducted at basic pH and hot temperature, preferentially at pH 10.0 and at 60° C.

The pH is then adjusted to a value comprised between 4.0 and 7.0, advantageously between 4.5 and 6.0. Sequential filtrations can be implemented until a 0.22 μm or 0.1 μm threshold, advantageously 0.22 μm. The obtained fraction is then evaporated and a dilution matrix can be added to the hydrolysate, in a final concentration in weight compared the volume of the matrix and the hydrolysate comprised between 2.5% and 10%, advantageously between 5% and 10%. Advantageously, the dilution matrix is chosen from propylene glycol, caprylyl glycol, butylene glycol, pentylene glycol, glycerin, still advantageously pentylene glycol.

In an advantageous embodiment of the invention, the hydrolysate according to the invention is a hydrolysate of the coproduct of a solid-liquid extraction of the dried biomass of a culture of P. tricornutum. Advantageously, the extraction is conducted with a solvent chosen from ethanol, methanol, isopropanol, a natural or branched oil, a glycol, a polyol, a mixture water/alcohol, a mixture water/glycol in the range 99/1 to 1/99 (w/v), more advantageously it is ethanol.

In a particular advantageous embodiment, the hydrolysate according to the invention is a hydrolysate obtained from the delipidated fraction (coproduct) issued from the solid-liquid extraction of the dried biomass of a culture of P. tricornutum with ethanol. The ethanolic extraction of the biomass is conducted from a biomass quantity of 10% in weight compared to the total volume of the biomass and ethanol. The delipidated fraction is rinsed with water and then filtrated. It is hydrolyzed under hot temperature (60° C.) under pH 10.0 and then adjusted to a pH of 6.0 and filtrated at 022 μm. The obtained hydrolysate is solubilized in pentylene glycol (5% w/v), under the conditions described in Example 1a).

In another preferential embodiment, the hydrolysate according to the invention is a hydrolysate obtained from the delipidated fraction (coproduct) issued from the solid-liquid extraction of the dried biomass of a culture of P. tricornutum with a mixture water/ethanol in proportion 30/70 (v/v) respectively. Said biomass extraction is conducted from a biomass quantity of 10% in weight compared to the total volume of the biomass and ethanol. The delipidated fraction is rinsed with water and then filtrated. It is hydrolyzed under hot temperature (60° C.) under pH 10.0 and then adjusted to a pH of 6.0 and filtrated at 022 μm. The obtained hydrolysate is solubilized in pentylene glycol (5% w/v), under the conditions described in Example 1b).

In yet another advantageous embodiment of the invention, the hydrolysate according to the invention is a hydrolysate obtained from the delipidated fraction (coproduct) issued from the extraction of the dried biomass of a culture of P. tricornutum under carbon dioxyde supercritical conditions, under a temperature of 50° C. and a pressure of 150 bar together with ethanol as a co-solvent (10%). The delipidated fraction is rinsed with water and then filtrated. It is hydrolyzed under hot temperature (60° C.) under pH 10.0. The obtained fraction is adjusted to pH 6.0 and then filtrated at 022 μm. The obtained hydrolysate is solubilized in pentylene glycol (5% w/v), under the conditions described in Example 1c).

The hydrolysate such as prepared in Examples 1a) to 1c) is under liquid form. It does not contain silica or organic silicium, under solid form, neither silicates, and only contain silicium under fully soluble form, advantageously under the form of orthosilicic acid.

The hydrolysate according to the invention can be used alone or in a composition.

In one embodiment of the invention, the hydrolysate is used in a cosmetic composition, advantageously non therapeutic, which comprises at least one cosmetic acceptable excipient.

The excipient is advantageously chosen from natural oils such as argan oil, shea oil, jojoba oil, avocado oil, sweet almond oil, preservatives, emulsifiers, emollients, surfactants, moisturizing agents, thickeners, texturizing agents, conditioners, gloss agents, texturizing agents, film-forming agents, pigments, coloring agents, perfumes, antimicrobial agents, biological additives, chelating agents, biocidal agents, astringent agents, polymers, reducing agents, pH regulators, humectants, conditioners.

The hydrolysate according to the invention is present in the cosmetic composition in a concentration in weight compared to the total weight of the composition comprised between 0.1% and 5% (w/w), advantageously between 1% and 2% (w/w).

The cosmetic composition is under the form of a cream, a serum, a gel, a soap, a dermatological bar, a shower gel, an aqueous or oily solution, an oil in water emulsion or a water in oil emulsion, a mask, a lotion, an ointment, a shampoo, a hair conditioner, a hair care, a foam. Advantageously, the composition is under the form of a cream or a serum. Therefore, the cosmetic composition is useful to exfoliate skin and/or to reduce skin microrelief, preferentially healthy skin, it is useful in order to increase skin cellular renewal.

In another embodiment of the invention, the hydrolysate of the extraction coproduct of P. tricornutum alone or in a composition comprising it, is used in a nutraceutical composition to exfoliate skin and/or to reduce skin microrelief. Said composition is advantageously non therapeutic and is intended for oral administration. The hydrolysate or the nutraceutical composition comprising it can therefore be used as a food supplement, with a nutra-cosmetic aim. The hydrolysate can be under liquid form or dried to obtain a powder.

The composition can be under the form of an emulsion and therefore comprises at least one acceptable nutraceutical excipient chosen from vegetal oils such as olive oil, canola oil, linseed oil, sunflower oil, grapeseed oil, palm oil, MCT oils (Medium Chain Triglycerides) said being composed of more than 70% in weight of a mixture of caprylic and capric acid, advantageously chosen from coco oil, palm oil, advantageously coco oil, more advantageously coconut oil.

The nutraceutical composition can comprise moreover one or more synthetic or natural antioxidants chosen from vitamin E or its derivatives, carotenoids, rosemary extract. It is meant here by “vitamin E” a tocopherol chosen from α-tocopherol, γ-tocopherol, β-tocopherol or δ-tocopherol, or a tocotrienol chosen from α-tocotrienol, β-tocotrienol, γ-tocotrienol or δ-tocotrienol. Advantageously, it is α-tocopherol.

Furthermore, the nutraceutical composition according to the invention comprises at least one additive chosen from preservative agents, colorants, flavors, disintegration agents, lubricant agents, coating or encapsulation agents.

The composition can be under powder form and is then under the form of gel capsules, capsules, pills, pastilles, liquid or loose powder. It is useful to exfoliate skin and/or to reduce skin microrelief, preferentially healthy, and to increase skin cellular renewal. The hydrolysate according to the invention is present in the nutraceutical composition in a concentration in weight compared to the total weight of the composition comprised in between 0.1% and 5% (w/w), advantageously between 1% and 2% (w/w).

The hydrolysate according to the invention can be in combination with any other cosmetic or nutraceutical (nutra-cosmetic) active having an exfoliation effect and/or an effect of reducing skin microrelief. The hydrolysate according to the invention can also be combined with any active ingredient having a complementary effect to the one of the extract, advantageously chosen from anti-aging, anti-wrinkle agents, increasing skin firmness, regenerating active ingredients, active ingredients onto sensitive skins, anti-pollution active ingredients or yet ingredients active onto skin microbiota. The hydrolysate according to the invention can also be combined to any lightening ingredient or active onto skin tone uniformity.

The hydrolysate according to the invention can furthermore be associated to one or more microalgae extracts chosen from an extract of Phaeodactylum tricornutum and/or an extract of Porphyridium cruentum, advantageously as an anti-aging active ingredient and protecting skin against urban stress and UV, and/or an extract of the microalgae Tisochrysis lutea, in particular as an active ingredient improving sensitive skin comfort.

An object of the invention still concerns a cosmetic care process, advantageously non therapeutic, comprising the oral administration or the topical application of a hydrolysate of an extraction coproduct of Phaeodactylum tricornutum. Very advantageously, the hydrolysate is being obtained by hydrolysis of an ethanolic extraction coproduct under basic pH, advantageously at pH 10.0 and under hot temperature, preferentially under a temperature of 60° C.

In an advantageous embodiment of the invention, the cosmetic care process comprises the topical application of the hydrolysate of the invention or of a cosmetic composition comprising it onto at least an area of skin, advantageously a healthy skin, chosen from any area of the face and/or any area of the arms, included forearms, legs, thighs, back, chest, neck, neckline, feet and/or hands, included scalp.

Examples referring to the description are presented below. These examples are illustrative and are not intended to limit the scope of the invention. The examples form an integral part of the present invention, and any new features relative to the prior art based on the description taken as a whole form an integral part of the invention.

Unless expressly mentioned, the temperature is given in degrees Celsius (C°), the pressure in bar, the percentages are expressed on a weight/weight basis (w/w). Ppm means Party per million (0.01%=100 ppm).

### LIST OF FIGURES

FIG. 1: Effect of the hydrolysate according to the invention on skin exfoliation (D0: time T0 of measurement of the squama quantity; D14 and D28: times of measurement of the squama quantity following 14 and 28 days of application of a cream comprising 1.5% (w/w) of the hydrolysate according to the invention (Ex. 1a) or of a placebo cream (Example 2).

FIG. 2: Effect of the hydrolysate according to the invention on the reduction of skin microrelief (D0: time T0 of measurement of skin microrelief; D28 hydrolysate: time 28 days following application of a cream comprising 1.5% (w/w) of the hydrolysate according to the invention (Ex. 1a); D28 placebo: time 28 days following application of a placebo cream without hydrolysate (Example 3).

### EXAMPLES

### Example 1: Methods of Preparation of a Hydrolysate of an Extraction Coproduct of Phaeodactylum tricornutum

The hydrolysate of the extraction coproduct is obtained from a dry biomass obtained from an autotrophic culture of a strain of P. tricornutum of French origin.

Example 1a) The dry biomass obtained from an autotrophic culture of the microalgae P. tricornutum in a photobioreactor under controlled conditions was extracted with ethanol (10% of biomass in weight compared to the total volume of solvent and biomass). The residue or coproduct of the extraction corresponding to the delipidated fraction was rinsed with water and filtrated. It was then hydrolyzed under basic pH (pH 10.0) and hot temperature (60° C.). The obtained fraction was adjusted to a pH of 6.0 and then filtrated until a 0.22 μm threshold. Pentylene glycol was added (5% weight/volume). The hydrolysate is under liquid form.

Example 1b) The dry biomass obtained from an autotrophic culture of the microalgae P. tricornutum in a photobioreactor under controlled conditions was extracted with a mixture water/ethanol 30/70 (v/v) (10% of biomass in weight compared to the total volume of solvent and biomass). The residue or coproduct of the extraction corresponding to the delipidated fraction was rinsed with water and filtrated. It was then hydrolyzed under basic pH (pH 10.0) and hot temperature (60° C.). The obtained fraction was adjusted to a pH of 6.0 and then filtrated until a 0.22 μm threshold. Pentylene glycol was added (5% w/v). The hydrolysate is under liquid form.

Example 1c) The dry biomass obtained from an autotrophic culture of the microalgae P. tricornutum in a photobioreactor under controlled conditions was extracted by supercritical carbon dioxide extraction with ethanol as co-solvent (10%), under a temperature of 50° C. and a pressure of 150 bars. The residue or coproduct of the extraction corresponding to the delipidated fraction was rinsed with water and filtrated. It was then hydrolyzed under basic pH (pH 10.0) and hot temperature (60° C.). The obtained fraction was adjusted to a pH of 6.0 and then filtrated until a 0.22 μm threshold. Pentylene glycol was added (5% w/v). The hydrolysate is under liquid form.

### Example 2: Exfoliating Effect of a Hydrolysate According to the Invention

Protocol: A clinical study was conducted onto a population of 35 healthy caucasian individuals, meaning not suffering from any cutaneous pathology, aged between 30 to 59 years old, with an average age of 49.3 years. A cream comprising 1.5% in weight of the hydrolysate according to the invention compared to the total weight of the cream (w/w) such as prepared in Example 1a), was applied twice a day during 28 consecutive days onto one of the forearms of the population. The same cream without hydrolysate as a placebo was applied under the same conditions on the second forearms.

The exfoliating effect was measured by a non-invasive technic allowing the sampling of epidermic superficial layers, using a patch made of a transparent acetate disk and an adhesive film named D'squame®. Said disk was glued onto the skin surface of the 2 forearms of the population, the one onto which the cream comprising the hydrolysate according to the invention was applied and the one onto which the placebo cream was applied. The disks were maintained during a period of 20 seconds onto the surface thanks to a standardized weight integrated into a push button. Said disks each allowed the sampling of 5 epidermic superficial layers notated G0, G1, G2, G3 et G4 (G4 being the deepest layer). The discs were positioned on a black surface with standardized lighting between shots, under a camera to enable image acquisition. The light was reflected by the presence of the epidermal layers removed with the patches. The whiter the image is, the greater is the amount of desquamated epidermal material.

Two parameters were measured: the surface area occupied by squama was measured in mm² using the image acquisition software (QuantiSquam®) and the desquamation index (DI) was calculated under the formulae described by previously cited Schatz et al. (1993):

$$ID = [ 2 \times A + \text{?} n \times ( n - 1 ) ] / 6 \qquad [ Math 1 ]$$

$$\text{?} \text{indicates text missing or illegible when filed}$$

where A corresponds to the total surface occupied by squama (mm²), Tn corresponds to the percentage of layers in relation to the thickness of each layer, and n corresponds to the thickness from 1 to 5.

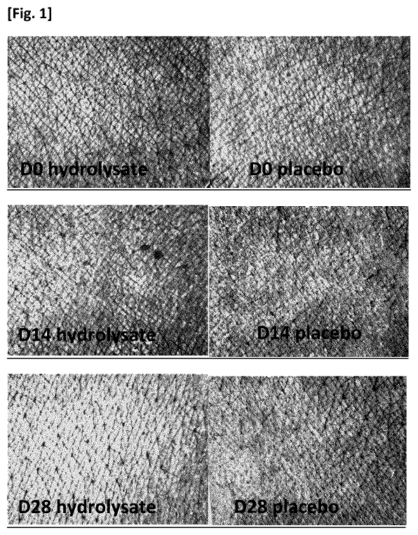

Results: the results are presented in Table 1 (Mean percentage (%) of the surface occupied (mm²)) and Table 2 (Mean (%) of the desquamation index (DI)) below (n=35) after 14 and 28 days of application, as well as in FIG. 1.

TABLE 1

| Surface occupied (mm²) | |
|---|---|
| (mean % of variation versus D 0) | % mean |
| D 14-D 0 Placebo cream | −4.95 |
| D 28-D 0 Placebo cream | −9.28 |
| D 14-D 0 Cream comprising 1.5% (w/w) | +2.01 |
| of the hydrolysate Example 1a) | |
| D 28-D 0 Cream comprising 1.5% (w/w) | +1.97 |
| of the hydrolysate Example 1a) | |

Conclusion: the cream comprising the hydrolysate of the invention did show its capacity to increase the mean surface occupied by squama compared to the placebo cream of more than 6.5% as from 14 days of application compared to the placebo, confirming the positive exfoliating effect of the hydrolysate of the invention onto skin.

TABLE 2

| Desquamation index (DI) | |
|---|---|
| (mean % of variation versus D 0) | % mean |
| D 14-D 0 Placebo cream | −2.48 |
| D 28-D 0 Placebo cream | −11.76 |
| D 14-D 0 Cream comprising 1.5% (w/w) | +8.68 |
| of the hydrolysate Example 1a) | |
| D 28-D 0 Cream comprising 1.5% (w/w) | +9.14 |
| of the hydrolysate Example 1a) | |

Conclusion: the cream comprising the hydrolysate did demonstrate its capacity to increase the desquamation index of the skin of the analyzed population of more than 10% compared to the placebo cream as from 14 days of application, and of more than 20% after 28 days, showing the positive effect of exfoliation of the hydrolysate of the invention onto skin over time and compared to the placebo.

### Example 3: Effect of Reduction of Skin Microrelief by a Hydrolysate According to the Invention

Protocol: the clinical study described in protocol of Example 2 was also conducted in order to evaluate the reduction of the skin microrelief with a hydrolysate according to the invention. A cream comprising the hydrolysate such as prepared in Example 1a) at a final concentration in weight compared to the total weight of the cream of 1.5%, was applied onto the first forearm of the healthy skin of the 35 individuals every day, twice a day, during a period of 28 days. The same cream comprising water instead of the hydrolysate was applied onto the second forearm under the same conditions (Placebo).

A silicon resin (Siflo®) was applied onto the forearms at TO (DO), after 28 days (D28) of application of the cream (or the placebo cream) and maintained on the skin during a period of 8 minutes in order to get the negative print of the analyzed portion of skin. Once dried, 3 parameters (Rt, Rz, Ra) were analyzed with the software Quantilines® (Cosderma). The Rt parameter corresponds to the surface heterogeneity, Rz corresponds to the skin rugosity and Ra corresponds to the skin relief.

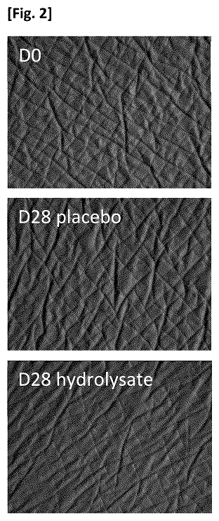

Results: results are presented in Table 3 below (mean % of variation of parameters Rt, Rz and Ra between D0 and D28 after application of a cream comprising 1.5% (w/w) of the hydrolysate of Example 1a) and in FIG. 2.

TABLE 3

| | Mean % of variation |
|---|---|
| | versus D 0 |
| Parameter Rt D 28-D 0 (1.5% | −2.70 |
| (w/w) hydrolysate example 1a) | |
| Parameter Rz D 28-D 0 (1.5% | −3.23 |
| (w/w) hydrolysate example 1a) | |
| Parameter Ra D 28-D 0 (1.5% | −2.77 |
| (w/w) hydrolysate example 1a) | |

Conclusion: the cream comprising 1.5% (w/w) of the hydrolysate of Example 1a) did allow the skin heterogeneity of the forearms of the studied population to decrease, as well as it reduced the skin rugosity and skin relief, after 28 days of application, demonstrating the efficacy of the hydrolysate onto the global reduction of the skin microrelief.

### Example 4: Examples of Cosmetic Formulations Comprising a Hydrolysate According to the Invention

The different phases of the formulations described in Table 5 below have been mixed under known methods by the one skilled in the art. Percentages are expressed in weight by total weight of the cream (w/w).

Example 4a) Cream comprising the hydrolysate of the invention Under ambient temperature, phase B was hydrated and mixed during 5 minutes. Sodium hydroxyde was added in order to neutralize pH (until 5.5). Phase D was added in the aqueous phase and the overall was mixed in order to form an emulsion (under ambient temperature, 5 minutes). Phases E, F, G and H were added and pH was adjusted to a pH comprised between 4.5 and 5.5.

TABLE 4

| PHASE | INGREDIENT | INCI NAME | % |
|---|---|---|---|
| A | Deionised water | Water | 78.84 |
| B | Carbopol ® Ultrez 10 polymer | Carbomer | 0.40 |
| C | Sodium hydroxyde | Sodium hydroxyde | 0.40 |
| D | PemuPur ™ START polymer | Microcristallin cellulose, fermented | 0.70 |
| | | extract of Shingomonas, cellulose gum | |
| D | MCT coco oil | Capryl/Capric Triglyceride | 7.00 |
| E | Zemea ® Propanediol | Propanediol | 10.00 |
| F | E-LEEN ® 8 | Caprylyl glycol, glycerin, water | 1.00 |
| G | Hydrolysate of the invention | Water, extract of Phaeodactylum | 1.50 |
| | | tricornutum, pentylene glycol | |
| H | Refreshing green | Perfume | 0.16 |
| I | Citric acid | Citric acid | QSP |

### Example 4b) Mask Comprising the Hydrolysate of the Invention

Under ambient temperature, phase B was pre-mixed with phase A during 5 minutes. Phase C was then added (5 minutes). Phases D and E were added one after the other under gentle agitation. Phase F was pre-mixed and added when the mixture was totally homogenized. Phase C was then added and pH was adjusted to a pH comprised between 4.5 and 5.5.

TABLE 5

| PHASE | INGREDIENT | INCI NAME | % |
|---|---|---|---|
| A | Deionised water | Water | 83.23 |
| B1 | Zemea ® Propanediol | Propanediol | 10.00 |
| B2 | Makimousse 25 | Sugar (Sodium Polyacrylate) | 2.50 |
| C | Covacryl MV 60 | Coco-caprylate/Caprate | 1.00 |
| D | E-LEEN ® 8 | Caprylyl glycol, glycerin, water | 1.00 |
| E | Hydrolysate of the invention | Water, extract of Phaeodactylum | 5.00 |
| | | tricornutum, pentylene glycol | |
| F | Refreshing green | Perfume | 0.30 |
| F | Cremophor ® RH 40 | Castor hydrogenated oil PEG-40 | 0.30 |
| G | Citric acid | Citric acid | QSP |

1.-14. (canceled)

15. A method of cosmetic care comprising the topical application to a human in need thereof of an effective amount of a hydrolysate of an extraction coproduct of Phaeodactylum tricornutum to exfoliate skin and/or to reduce skin microrelief.

16. The method according to claim 15 wherein the hydrolysate is in a cosmetic composition comprising at least one cosmetic acceptable excipient, and wherein the hydrolysate is present in the cosmetic composition in an amount comprised between 0.1% and 5%, advantageously between 1% and 2%, in weight compared to the total weight of the composition.

17. The method according to claim 16 wherein the cosmetic composition is under the form of a cream, a serum, a gel, a soap, a dermatological bar, a shower gel, an aqueous or oily solution, an emulsion oil in water or water in oil, a mask, a lotion, an ointment, a shampoo, a hair conditioner, a hair care, a foam.

18. The method according to claim 15 to increase skin cellular renewal.

19. The method according to claim 15 wherein the hydrolysate is a hydrolysate of an extraction coproduct of Phaeodactylum tricornutum by supercritical carbon dioxide or with a solvent chosen from ethanol, methanol, isopropanol, a natural or branched oil, a glycol, a polyol, a mixture water/alcohol, water/glycol in a ratio from 99/1 to 1/99 (w/v).

20. The method according to claim 19 wherein the hydrolysate is a hydrolysate of an ethanolic extraction coproduct of Phaeodactylum tricornutum.

21. The method according to claim 20 wherein the hydrolysate is obtained by hot hydrolysis under basic pH.

22. The method according to claim 15 comprising the topical application of the hydrolysate of an extraction coproduct of Phaeodactylum tricornutum or of a composition comprising it on at least a portion of skin chosen from any part of the face and/or any part of the arms, included forearms, legs, thighs, back, chest, neck, neckline, feet and/or hands, included scalp.

23. A method of nutraceutical care comprising the oral administration to a human in need thereof of an effective amount of a hydrolysate of an extraction coproduct of Phaeodactylum tricornutum alone or in a composition comprising it to exfoliate skin and/or to decrease skin microrelief.

24. The method according to claim 23 wherein the hydrolysate is present in the composition in a concentration between 0.1% and 5%, advantageously between 1% and 2%, in weight compared to the total weight of the composition.

25. The method according to claim 23 to increase skin cellular renewal.

26. The method according to claim 23 wherein the hydrolysate is a hydrolysate of an extraction coproduct of Phaeodactylum tricornutum by supercritical carbon dioxide or with a solvent chosen from ethanol, methanol, isopropanol, a natural or branched oil, a glycol, a polyol, a mixture water/alcohol, water/glycol in a ratio from 99/1 to 1/99 (w/v).

27. The method according to claim 26 wherein the hydrolysate is a hydrolysate of an ethanolic extraction coproduct of Phaeodactylum tricornutum.

28. The method according to claim 27 wherein the hydrolysate is obtained by hot hydrolysis under basic pH.
